(19) United States
(12) Patent Application Publication
(10) Pub. No.: US 2022/0047592 A1
(43) Pub. Date: Feb. 17, 2022

(54) METHODS OF TREATING RESPIRATORY DISEASES USING C5a INHIBITORS

(71) Applicant: CHEMOCENTRYX, INC., San Carlos, CA (US)

(72) Inventor: Markus J. CAPPEL, San Carlos, CA (US)

(21) Appl. No.: 17/400,257

(22) Filed: Aug. 12, 2021

Related U.S. Application Data
(60) Provisional application No. 63/064,991, filed on Aug. 13, 2020.

(51) Int. Cl. A61K 31/506; A61K 31/445; A61K 31/437; A61K 31/4162; A61P 31/14

(52) U.S. Cl. CPC A61K 31/506; A61K 31/445; A61P 31/14; A61K 31/4162; A61K 31/437

(57) ABSTRACT

Provided herein are methods of treating a respiratory disease in a subject in need thereof by administering an effective amount of a small molecule C5a inhibitor. Also provided herein are methods of treating coronavirus disease 2019 (COVID-19) in a subject in need thereof by administering an effective amount of a small molecule C5a inhibitor. In some embodiments, the small molecule C5a inhibitor is a compound of Formula (I), Formula (II), Formula (III), Formula (IV) or an embodiment described herein.

### CROSS-REFERENCES TO RELATED APPLICATIONS

This application claims the benefit of priority under 35 U.S.C. § 119(e) to U.S. Provisional Application Ser. No. 63/064,991 filed Aug. 13, 2020, the disclosure of which is incorporated herein by reference in its entirety.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BRIEF SUMMARY OF THE INVENTION

In some aspects, provided herein are methods for treating a respiratory disease in a subject comprising administering to the subject an effective amount of a small molecule C5a inhibitor. Exemplary small molecule C5a inhibitors are described in Section B. in the Detailed Description of this application.

In some aspects, provided herein are methods of treating coronavirus disease 2019 (COVID-19) in a subject comprising administering to the subject an effective amount of a small molecule C5a inhibitor. Exemplary small molecule C5a inhibitors are described in Section B. in the Detailed Description of this application.

### BRIEF DESCRIPTION OF THE DRAWINGS

Not Applicable

### DETAILED DESCRIPTION OF THE INVENTION

### I. Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. The term “heterocycloalkyl” refers to a cycloalkyl group that contain from one to five heteroatoms selected from N, O, and S, wherein the nitrogen and sulfur atoms are optionally oxidized, and the nitrogen atom(s) are optionally quaternized. The heterocycloalkyl may be a monocyclic, a bicyclic or a polycylic ring system. Non limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, and the like. A heterocycloalkyl group can be attached to the remainder of the molecule through a ring carbon or a heteroatom.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms. Similarly, “alkenylene” and “alkynylene” refer to the unsaturated forms of “alkylene” having double or triple bonds, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the terms “heteroalkenyl” and “heteroalkynyl” by itself or in combination with another term, means, unless otherwise stated, an alkenyl group or alkynyl group, respectively, that contains the stated number of carbons and having from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group.

The term “heteroalkylene” by itself or as part of another substituent means a divalent radical, saturated or unsaturated or polyunsaturated, derived from heteroalkyl, as exemplified by —CH₂—CH₂—S—CH₂CH₂— and —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like).

The above terms (e.g., “alkyl,” “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below, while the term “alkyl” and related aliphatic radicals is meant to refer to unsubstituted version, unless indicated to be substituted.

Substituents for the alkyl radicals (including those groups often referred to as alkylene, alkenyl, alkynyl and cycloalkyl) can be a variety of groups selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —CN and —NO₂ in a number ranging from zero to (2 m′+1), where m′ is the total number of carbon atoms in such radical. R′, R″ and R′″ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl” as used by itself or as part of another group refers to an alkyl radical wherein two substitutents on the carbon that is closest to the point of attachment for the radical is replaced with the substitutent ═O (e.g., —C(O)CH₃, —C(O)CH₂CH₂OR′ and the like).

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}-U-, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### II. Detailed Description of Embodiments

A. Treating Respiratory Diseases

In some aspects, provided herein are methods for treating a respiratory disease in a subject comprising administering to the subject an effective amount of a small molecule C5a inhibitor. Exemplary small molecule C5a inhibitors are described in Section B. of this application.

In some embodiments, the respiratory disease is caused by a virus.

In some embodiments, the virus is selected from the group consisting of an influenza virus, a coronavirus, a respiratory syncytial virus.

In some embodiments, the influenza virus is selected from the group consisting of influenza A virus, influenza B virus, and influenza C virus. In some embodiments, the influenza A virus is selected from the group consisting of H1N1, H5N1, and H7N9.

In some embodiments, the coronavirus is selected from the group consisting of severe acute respiratory syndrome coronavirus (SARS-CoV), severe acute respiratory syndrome coronavirus 2 (SARS-CoV-2), and middle east respiratory syndrome (MERS) coronavirus.

In some embodiments, the respiratory disease is acute lung injury (ALI). In some embodiments, the respiratory disease is acute respiratory distress syndrome (ARDS). In some embodiments, the respiratory disease is severe acute respiratory syndrome (SARS).

In some aspects, provided herein are methods of treating coronavirus disease 2019 (COVID-19) in a subject comprising administering to the subject an effective amount of a small molecule C5a inhibitor. Exemplary small molecule C5a inhibitors are described in Section B. of this application.

B. Small Molecule C5a Inhibitors

A number of small molecule C5a inhibitors are suitable for the present disclosure. Useful C5a inhibitors include but are not limited to compounds of Formula (I), Formula (II), Formula (III), and Formula (IV).

In some embodiments, the small molecule C5a inhibitor is a compound of Formula (I)

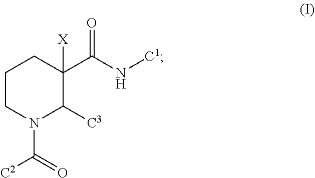

and pharmaceutically acceptable salts, hydrates and rotomers thereof, wherein
C¹ is selected from the group consisting of aryl and heteroaryl, wherein the heteroaryl group has from 1-3 heteroatoms as ring members selected from N, O and S; and wherein said aryl and heteroaryl groups are optionally substituted with from 1 to 3 R¹ substituents;
C² is selected from the group consisting of aryl and heteroaryl, wherein the heteroaryl group has from 1-3 heteroatoms as ring members selected from N, O and S; and wherein said aryl and heteroaryl groups are optionally substituted with from 1 to 3 R² substituents;
C³ is selected from the group consisting of C₁₋₈ alkyl, C₃₋₈ cycloalkyl, C₃₋₈ cycloalkyl-C₁₋₄ alkyl, aryl, aryl-C₁₋₄ alkyl, heteroaryl, heteroaryl-C₁₋₄ alkyl, heterocycloalkyl or heterocycloalkyl-C₁₋₄ alkyl, wherein the heterocycloalkyl group or portion has from 1-3 heteroatoms selected from N, O and S, and wherein the heteroaryl group has from 1-3 heteroatoms as ring members selected from N, O and S, and each C³ is optionally substituted with from 1-3 R³ substituents;
each R¹ is independently selected from the group consisting of
   halogen, —CN, —R^{c}, —CO₂R^{a}, —CONR^{a}R^{b}, —C(O)R^{a}, —OC(O)NR^{a}R^{b}, —N^{b}C(O)R^{a}, —NR^{b}C(O)₂R, —NR^{a}—C(O)NR^{a}R^{b}, —NR^{a}C(O)N^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₃₋₆ cycloalkyl, heterocycloalkyl, aryl and heteroaryl, and wherein the aliphatic and cyclic portions of R^{a}, R^{b} and R^{c} are optionally further substituted with from one to three halogen, hydroxy, methyl, amino, alkylamino and dialkylamino groups; and optionally when two R¹ substituents are on adjacent atoms, are combined to form a fused five or six-membered carbocyclic ring;
each R² is independently selected from the group consisting of
   halogen, —CN, —R^{f}, —CO₂R^{d}, —CONR^{d}R^{e}, —C(O)R^{d}, —OC(O)NR^{d}R^{e}, —NR^{c}(O)R^{d}, —NR^{e}C(O)₂R^{f}, —NR^{d}C(O)NR^{d}R^{e}, —NR^{d}C(O)NR^{d}R^{e}, —NR^{d}R^{e}, —OR^{d}, and —S(O)₂NR^{d}R^{c}; wherein each R^{d} and R^{e} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S; each R^{f} is independently selected from the group consisting of C₁₋₈ alkyl, C₁₋₈ haloalkyl, C₃₋₆ cycloalkyl, heterocycloalkyl, aryl and heteroaryl, and wherein the aliphatic and cyclic portions of R^{d}, R^{e} and R^{f} are optionally further substituted with from one to three halogen, hydroxy, methyl, amino, alkylamino and dialkylamino groups;
each R³ is independently selected from the group consisting of
   halogen, —CN, —Rⁱ, —CO₂R^{g}, —CONR^{g}R^{h}, —C(O)R^{g}, —OC(O)NR^{g}R^{h}, —NR^{h}C(O)R^{g}, —NR^{h}C(O)₂Rⁱ, —NR^{g}(O)NR^{g}R^{h}, —NR^{g}R^{h}, —OR^{g}, —S(O)₂NR^{g}R^{h}, —X⁴—R^{j}, —X⁴—NR^{g}R^{h}, —X⁴—CONR^{g}R^{h}, —X⁴—NR^{h}C(O)R^{g}, —NHR^{j} and —NHCH₂R^{j}, wherein X⁴ is a C₁₋₄ alkylene; each R^{g} and R^{h} is independently selected from hydrogen, C₁₋₈ alkyl, C₃₋₆ cycloalkyl and C₁₋₈ haloalkyl, or when attached to the same nitrogen atom can be combined with the nitrogen atom to form a five or six-membered ring having from 0 to 2 additional heteroatoms as ring members selected from N, O or S and is optionally substituted with one or two oxo; each R is independently selected from the group consisting of C₁₋₈ alkyl, C₁₋₈ haloalkyl, C₃₋₆ cycloalkyl, heterocycloalkyl, aryl and heteroaryl; and each R^{j} is selected from the group consisting of C₃₋₆ cycloalkyl, pyrrolinyl, piperidinyl, morpholinyl, tetrahydrofuranyl, and tetrahydropyranyl, and wherein the aliphatic and cyclic portions of R^{g}, R^{h}, Rⁱ and R^{j} are optionally further substituted with from one to three halogen, methyl, CF₃, hydroxy, amino, alkylamino and dialkylamino groups; and
X is hydrogen or CH₃.

In some embodiments, compounds of formula I have subformula Ie:

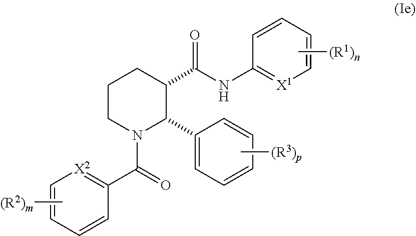

wherein the subscript p is an integer of from 0 to 3; X¹ is selected from the group consisting of N, CH and CR¹; the subscript n is an integer of from 0 to 2; X² is selected from the group consisting of N, CH and CR²; and the subscript m is an integer of from 0 to 2.

In some embodiments, the compound of Formula (I) has the structure

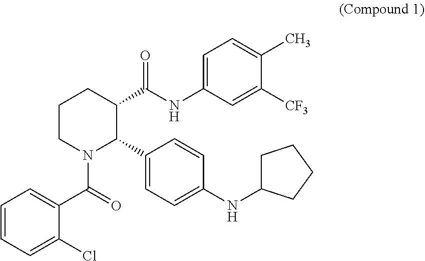 [SMILES: Cc1ccc(NC(=O)[C@H]2CCCN(C(=O)c3ccccc3Cl)[C@H]2c2ccc(NC3CCCC3)cc2)cc1C(F)(F)F]

or a pharmaceutically acceptable salt thereof.

In some embodiments, the compound of Formula (I) has the structure

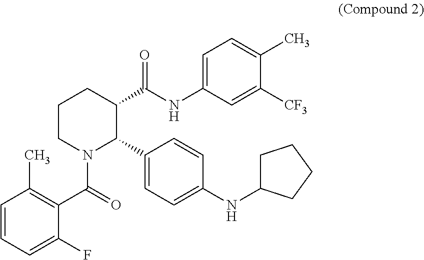 [SMILES: Cc1ccc(NC(=O)[C@H]2CCCN(C(=O)c3c(C)cccc3F)[C@H]2c2ccc(NC3CCCC3)cc2)cc1C(F)(F)F]

or a pharmaceutically acceptable salt thereof.

In some embodiments, the compound of Formula (I) has the structure

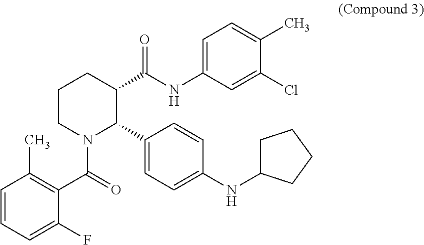 [SMILES: Cc1ccc(NC(=O)[C@H]2CCCN(C(=O)c3c(C)cccc3F)[C@H]2c2ccc(NC3CCCC3)cc2)cc1Cl]

or a pharmaceutically acceptable salt thereof.

In some embodiments, the compound of Formula (I) has the structure

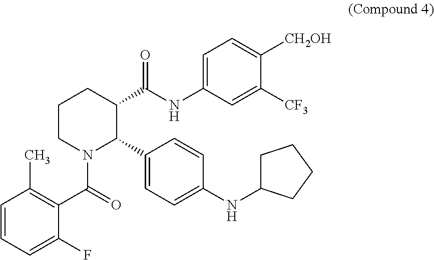 [SMILES: Cc1cccc(F)c1C(=O)N1CCC[C@H](C(=O)Nc2ccc(CO)c(C(F)(F)F)c2)[C@@H]1c1ccc(NC2CCCC2)cc1]

or a pharmaceutically acceptable salt thereof.

In some embodiments, the C5a inhibitor is selected from the formulas, compounds or pharmaceutical compositions disclosed in WO2011/163640 (PCT Application No. PCT/US2011/041910, filed on Jun. 24, 2011 by ChemoCentryx), the content of which is incorporated herein for all purposes.

In some embodiments, the small molecule C5a inhibitor is a compound of Formula (II)

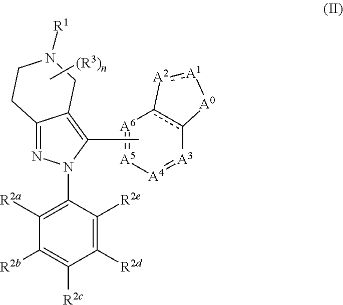

or a pharmaceutically acceptable salt thereof, wherein,
ring vertex A⁰ is NH or C(O);
each of ring vertices A¹ and A³ are independently selected from the group consisting of N, NH, CH, C(O) and C(R⁴);
each of ring vertices A², A⁵ and A⁶ is independently selected from the group consisting of N, CH, and C(R⁴);
ring vertex A⁴ is selected from the group consisting of N, N(C₁₋₄ alkyl), CH, and C(R⁴); and no more than two of A³, A⁴, A⁵ and A⁶ are N;
each of the dashed bonds independently is a single or double bond;
R¹ is selected from the group consisting of heteroaryl, C₆₋₁₀ aryl, —C₁₋₈ alkylene-heteroaryl, —C₁₋₈ alkylene-C₆₋₁₀ aryl, C₃₋₈ cycloalkyl, four to eight membered heterocycloalkyl, C₁₋₈ alkyl, C₁₋₈ haloalkyl, —C(O)NR^{1a}R^{1b}, and —CO₂R^{1a}; wherein the heterocycloalkyl group is a 4 to 8 membered ring having from 1 to 3 heteroatoms as ring vertices selected from N, O and S; the heteroaryl group is a 5 to 10 membered aromatic ring having from 1 to 3 heteroatoms as ring vertices selected from N, O and S;
wherein R^{1a} and R^{1b} are each independently selected from the group consisting of hydrogen, C₁₋₈ alkyl, C₆₋₁₀ aryl, and —C₁₋₆ alkylene-C₆₋₁₀ aryl;
wherein R¹ is optionally substituted with 1 to 5 R⁵ substituents;
R^{2a} and R^{2e} are each independently selected from the group consisting of C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, CN, and halogen;
R^{2b}, R^{2c}, and R^{2d} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, cyano, and halogen;
each R³ is independently selected from the group consisting of C₁₋₄ alkyl, C₁₋₄ haloalkyl and hydroxyl, and optionally two R³ groups on the same carbon atom are combined to form oxo (═O);
each R⁴ is independently selected from the group consisting of C₁₋₆ alkyl, C₁₋₆ alkoxy, C₁₋₆ hydroxyalkyl, C₁ haloalkyl, C₁₋₆ haloalkoxy, —O—C₁₋₆ haloalkyl, halogen, cyano, hydroxyl, —S—C₁₋₆ alkyl, —C₁₋₆ alkyl-O—C₁₋₆ alkyl, —C₁₋₆ alkyl-S—C₁₋₆ alkyl, —NR^{4a}R^{4b}, —CONR^{4a}R^{4b}, —CO₂R^{4a}, —COR^{4a}, —OC(O)NR^{4a}R^{4b}, —NR^{4a}C(O)R^{4b}, —NR^{4a}C(O)₂R^{4b}, and —NR^{4a}—C(O)NR^{4a}R^{4b};
each R^{4a} and R^{4b} is independently selected from the group consisting of hydrogen, C₁₋₆ alkyl, and C₁₋₆ haloalkyl;
each R⁵ is independently selected from the group consisting of C₁₋₈ alkyl, C₁₋₈ alkoxy, C₁₋₈ haloalkyl, C₁₋₈ haloalkoxy, C₁₋₈ hydroxyalkyl, —C₁₋₈ alkyl-heterocycloalkyl, —C₁₋₈ alkyl-C₃₋₈ cycloalkyl, C₃₋₆ cycloalkyl, heterocycloalkyl, halogen, OH, C₂₋₈ alkenyl, C₂₋₈ alkynyl, CN, C(O)R^{5a}, —NR^{5b}C(O)R^{5a}, —CONR^{5a}R^{5b}, —NR^{5a}R^{5b}, —C₁₋₈ alkylene-NR^{5a}R^{5b}, —S—C₁₋₆ alkyl, —C₁₋₆ alkyl-O—C₁₋₆ alkyl, —C₁₋₆ alkyl-S—C₁₋₆ alkyl, —OC(O)NR^{5a}R^{5b}, —NR^{5a}C(O)₂R^{5b}, —NR^{5a}—C(O)NR^{5b}R^{5b} and CO₂R^{5a}; wherein wherein the heterocycloalkyl group is a 4 to 8 membered ring having from 1 to 3 heteroatoms as ring vertices selected from N, O and S;
wherein each R^{5a} and R^{5b} is independently selected from the group consisting of hydrogen, C₁₋₄ alkyl, and C₁₋₄ haloalkyl, or when attached to the same nitrogen atom R^{5a} and R^{5b} are combined with the nitrogen atom to form a five or six-membered ring having from 0 to 1 additional heteroatoms as ring vertices selected from N, O, or S; and
the subscript n is 0, 1, 2 or 3.

In some embodiments, the compound of Formula (II) is represented by Formula (IIf), (IIg), (IIh), or (IIi)

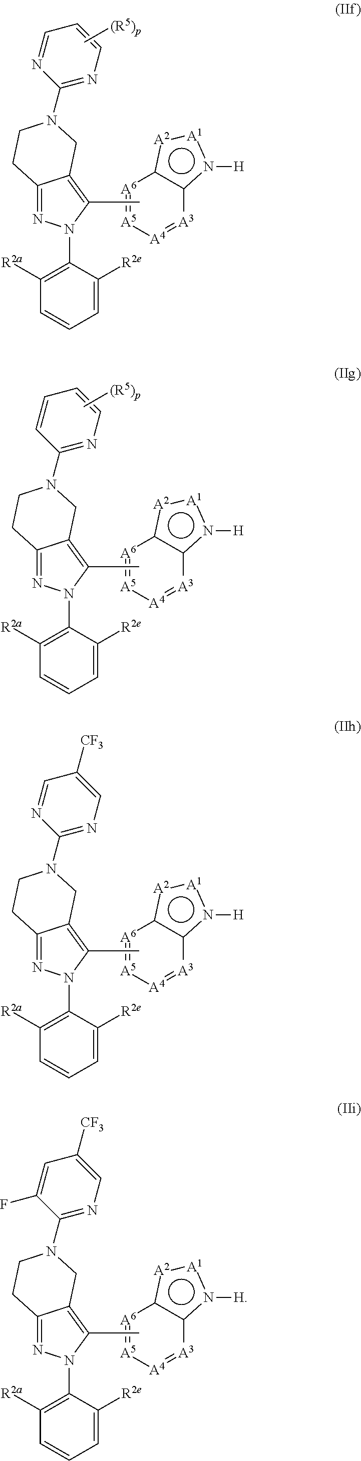

In embodiments where the compound of Formula (II) is represented by Formula (IIf), (IIg), (IIh), or (IIi), the ring portion having A¹, A², A³, A⁴, A³, and A⁶ as ring vertices, R^{2a}, R^{2e}, and R⁵ are as defined above for Formula (II), and p is 0, 1 or 2.

In some embodiments, the compound of Formula (II) has the structure

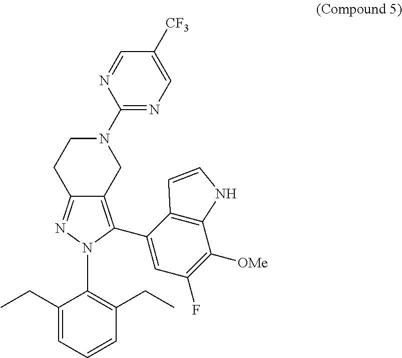 [SMILES: CCc1cccc(CC)c1-n1nc2c(c1-c1cc(F)c(OC)c3[nH]ccc13)CN(c1ncc(C(F)(F)F)cn1)CC2]

or a pharmaceutically acceptable salt thereof.

In some embodiments, the compound of Formula (II) has the structure

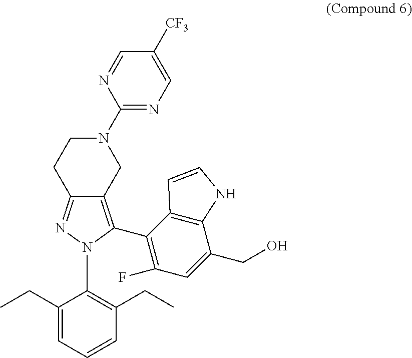 [SMILES: CCc1cccc(CC)c1-n1nc2c(c1-c1c(F)cc(CO)c3[nH]ccc13)CN(c1ncc(C(F)(F)F)cn1)CC2]

or a pharmaceutically acceptable salt thereof.

In some embodiments, the compound of Formula (II) has the structure

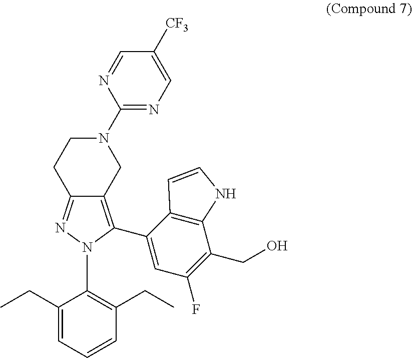 [SMILES: CCc1cccc(CC)c1-n1nc2c(c1-c1cc(F)c(CO)c3[nH]ccc13)CN(c1ncc(C(F)(F)F)cn1)CC2]

or a pharmaceutically acceptable salt thereof.

In some embodiments, the compound of Formula (II) has the structure

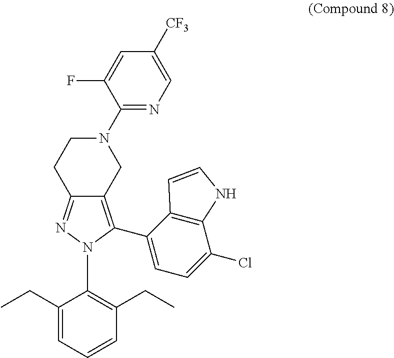 [SMILES: CCc1cccc(CC)c1-n1nc2c(c1-c1ccc(Cl)c3[nH]ccc13)CN(c1ncc(C(F)(F)F)cc1F)CC2]

or a pharmaceutically acceptable salt thereof.

In some embodiments, the C5a inhibitors selected from the formulas, compounds or pharmaceutical compositions disclosed in WO2018/222598 (PCT Application No. PCT/US2018/34905, filed on May 29, 2018 by ChemoCentryx), the content of which is incorporated herein for all purposes.

In some embodiments, the small molecule C5a inhibitor is a compound of Formula (III)

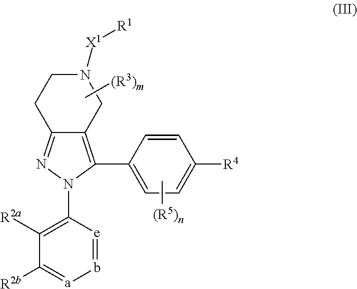

or a pharmaceutically acceptable salt thereof, wherein,
ring vertex a is N or C(R^{2c}), ring vertex b is N or C(R^{2d}), and ring vertex e is N or C(R^{2e}), wherein no more than one of a, b and e is N;
X¹ is selected from the group consisting of a bond, C₁₋₈ alkylene, C(O), C(O)—C₁₋₄ alkylene, and S(O)₂;
R¹ is selected from the group consisting of
   a) 5- to 10-membered heteroaryl having from 1 to 4 heteroatoms as ring vertices selected from N, O and S;
   b) C₆₋₁₀ aryl;
   c) C₃₋₈ cycloalkyl;
   d) 4- to 8-membered heterocycloalkyl having from 1 to 2 heteroatoms as ring vertices selected from N, O and S; and
   e) C₁₋₈ alkyl, C₁₋₈ alkoxy, C₁₋₈ haloalkyl, —C(O)NR^{1a}R^{1b}, and —CO₂R^{1a}; wherein R^{1a} and R^{1b} are each independently selected from the group consisting of hydrogen, C₁₋₈ alkyl, C₆₋₁₀ aryl, and —C₁₋₆ alkylene-C₆₋₁₀ aryl;
wherein the group —X¹—R¹ is optionally substituted with 1 to 5 R^{x} substituents;
R^{2a} and R^{2e} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C¹⁻⁶ alkyl, CN, and halogen, and at least one of R^{2a} and R^{2b} is other than hydrogen;
R^{2b}, R^{2c}, and R^{2d} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, cyano, and halogen;
each R³ is independently selected from the group consisting of hydroxyl, C₁₋₄ alkyl, C₁₋₄ haloalkyl and C₁₋₄ hydroxyalkyl, and optionally two R³ groups on the same carbon atom are combined to form oxo (═O), and optionally two R³ groups and the carbon atoms they are attached to form a 3-6 membered ring with 0-2 hetereoatoms as ring members selected from O, N, and S;
R⁴ is independently selected from the group consisting of —X²—OR^{4a}, —X²—NR^{4a}R^{4b}, —X²—CONR^{4a}R^{4b}, —X²—NR^{4a}—C(O)R^{4a}, —X²—NR^{4a}—C(O)NR^{4a}R^{4b}, —X²—N^{4a}—C(O)OR^{4a}, —X²—NR^{4a}—C(O)—C₁₋₃ alkylene-OR^{4a} and —X²—NR^{4a}—C(O)—C₁₋₃ alkylene-NR^{4a}R^{4b}; wherein each X² is independently a bond, C(O), C₁₋₄ alkylene, C(O)—C₁₋₄ alkylene, and C₁₋₄ alkylene-C(O), and each R^{4a} and R^{4b} is independently selected from the group consisting of hydrogen, C₁₋₄ alkyl, and C₁₋₄ haloalkyl;
each R⁵ is independently selected from the group consisting of C₁₋₈ alkyl, C₁₋₈ alkoxy, C₁₋₈ haloalkyl, C₁₋₈ haloalkoxy, C₁₋₈ hydroxyalkyl, halogen, OH, CN, C(O)R^{5a} and CO₂R^{5a}; wherein each R^{5a} is independently selected from the group consisting of hydrogen, C₁₋₄ alkyl, and C₁₋₄ haloalkyl;
each R^{x} is independently selected from the group consisting of halogen, CN, C₁₋₄ alkyl, C₁₋₄ alkoxy, C₁₋₄ haloalkyl, C₁₋₄ haloalkoxy, C₁₋₄ hydroxy, C₂₋₄ alkenyl, C₃₋₆ cycloalkyl, CO₂—C₁₋₄ alkyl, and CONH₂;
the subscript m is 0, 1, 2, 3 or 4; and
the subscript n is 0, 1, 2 or 3.

With reference to the compounds of formula (III), or a pharmaceutically acceptable salt thereof, as well as any of the embodiments noted above, in some further embodiments, the group

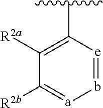

is selected from the group consisting of

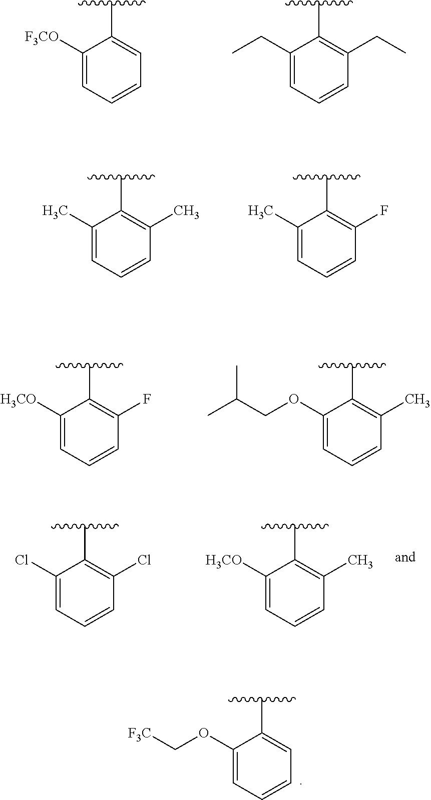

In some embodiments, the compound of Formula (III) has the structure

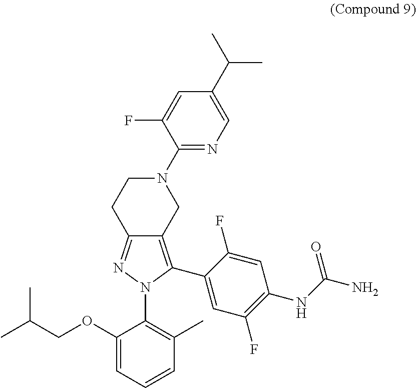 [SMILES: Cc1cccc(OCC(C)C)c1-n1nc2c(c1-c1cc(F)c(NC(N)=O)cc1F)CN(c1ncc(C(C)C)cc1F)CC2]

or a pharmaceutically acceptable salt thereof.

In some embodiments of the compounds of formula (III), or a pharmaceutically acceptable salt thereof, —X¹—R¹ is selected from the group consisting of:

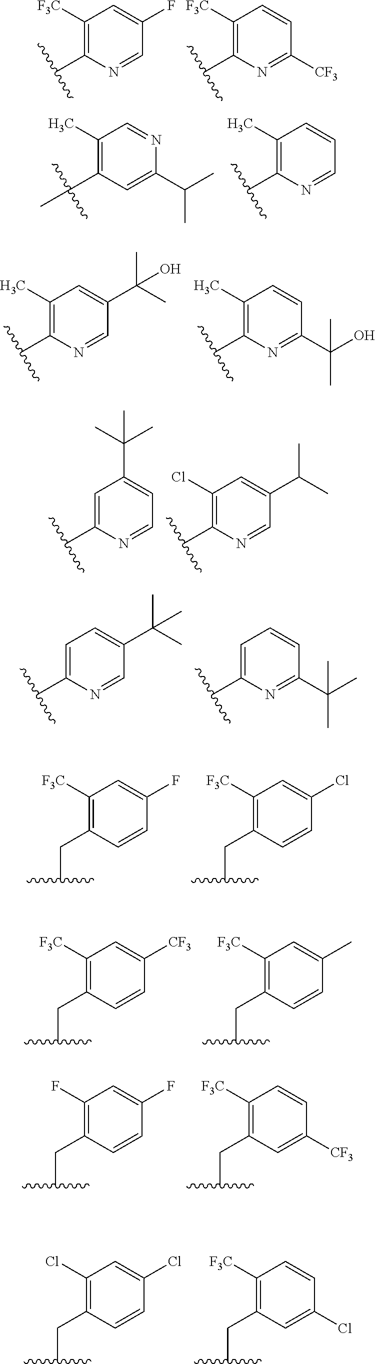

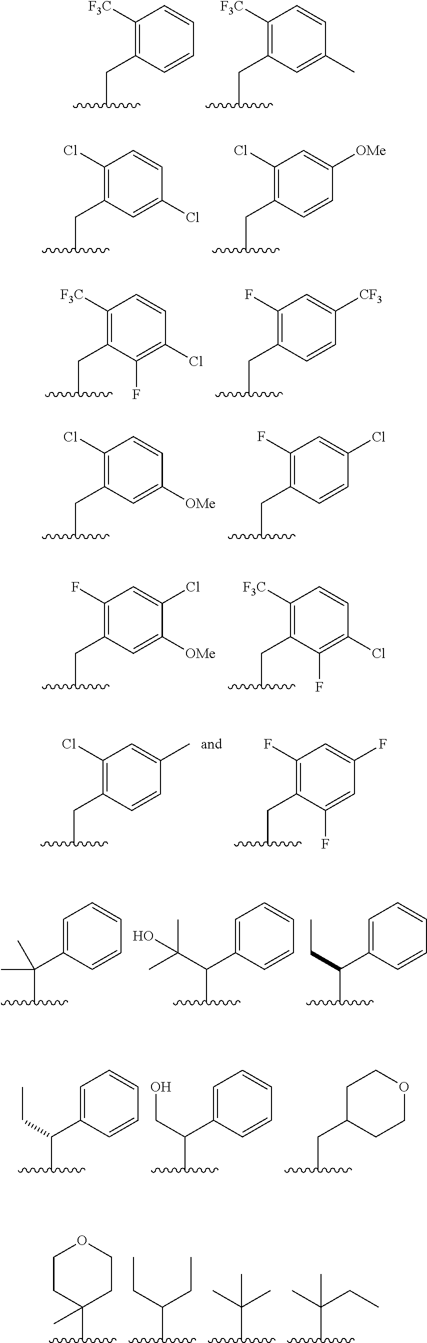

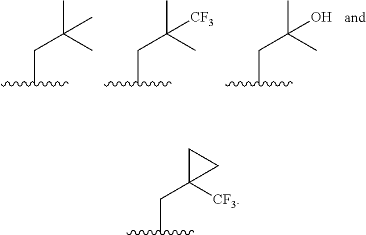

In some embodiments, the compound of Formula (III) has the structure

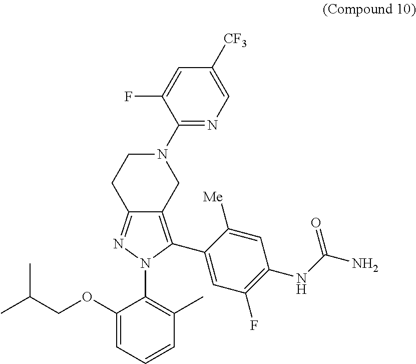 [SMILES: Cc1cc(NC(N)=O)c(F)cc1-c1c2c(nn1-c1c(C)cccc1OCC(C)C)CCN(c1ncc(C(F)(F)F)cc1F)C2]

or a pharmaceutically acceptable salt thereof.

In some embodiments, the compound of Formula (III) has the structure

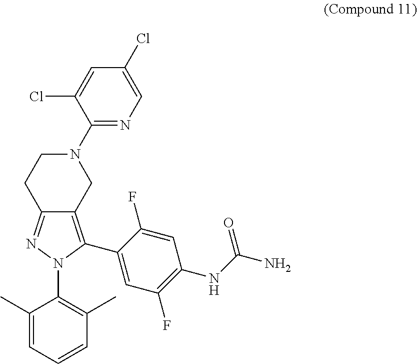 [SMILES: Cc1cccc(C)c1-n1nc2c(c1-c1cc(F)c(NC(N)=O)cc1F)CN(c1ncc(Cl)cc1Cl)CC2]

or a pharmaceutically acceptable salt thereof.

In some embodiments, the compound of Formula (III) has the structure

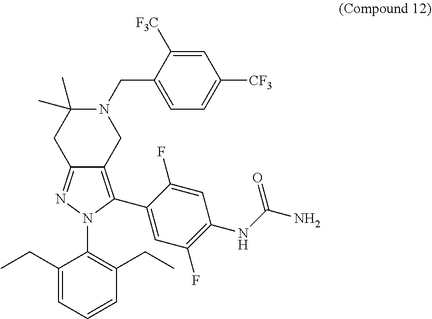 [SMILES: CCc1cccc(CC)c1-n1nc2c(c1-c1cc(F)c(NC(N)=O)cc1F)CN(Cc1ccc(C(F)(F)F)cc1C(F)(F)F)C(C)(C)C2]

or a pharmaceutically acceptable salt thereof.

In some embodiments, the C5a inhibitor is selected from the formulas, compounds or pharmaceutical compositions disclosed in WO2019/126424 (PCT Application No. PCT/US2018/66667, filed on Dec. 20, 2018 by ChemoCentryx), the content of which is incorporated herein for all purposes.

In some embodiments, the small molecule C5a inhibitor is a compound of Formula (IV)

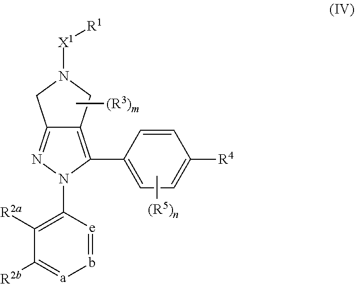

or a pharmaceutically acceptable salt thereof, wherein,
ring vertex a is N or C(R^{2c}), ring vertex b is N or C(R^{2d}), and ring vertex e is N or C(R^{2c}), wherein no more than one of a, b and e is N;
X¹ is selected from the group consisting of a bond, C₁₋₈ alkylene, C(O), C(O)—C₁₋₄ alkylene, and S(O)₂;
R¹ is selected from the group consisting of
   a) 5- to 10-membered heteroaryl having from 1 to 4 heteroatoms as ring vertices selected from N, O and S;
   b) C₆₋₁₀ aryl;
   c) C₃₋₈ cycloalkyl;
   d) 4- to 8-membered heterocycloalkyl having from 1 to 2 heteroatoms as ring vertices selected from N, O and S; and
   e) C₁₋₈ alkyl, C₁₋₈ alkoxy, C₁₋₈ haloalkyl, —C(O)NR^{1a}R^{1b}, and —CO₂R^{1a}; wherein R^{1a} and R^{1b} are each independently selected from the group consisting of hydrogen, C₁₋₈ alkyl, C₆₋₁₀ aryl, and —C₁₋₆ alkylene-C₆₋₁₀ aryl;
wherein the group —X¹—R¹ is optionally substituted with 1 to 5 R^{x} substituents;
R^{2a} and R^{2e} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, CN, and halogen, and at least one of R^{2a} and R^{2e} is other than hydrogen;
R^{2b}, R^{2c}, and R^{2d} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, cyano, and halogen;
each R³ is independently selected from the group consisting of hydroxyl, C₁₋₄ alkyl, C₁₋₄ haloalkyl and C₁₋₄ hydroxyalkyl, and optionally two R³ groups on the same carbon atom are combined to form oxo (═O), and optionally two R³ groups and the carbon atoms they are attached to form a 3-6 membered ring with 0-2 hetereoatoms as ring members selected from O, N, and S;
R⁴ is independently selected from the group consisting of —X²—OR^{4a}, —X²—NR^{4b}, —X²—CONR^{4a}R^{4b}, —X²—NR^{4a}—C(O)R^{4a}, —X²—NR^{4a}—C(O)NR^{4a}R^{4b}, —X²—NR^{4a}—C(O)OR^{4a}, —X²—NR^{4a}—C(O)—C₁₋₃ alkylene-OR^{4a} and —X²—NR^{4a}—C(O)—C₁₋₃ alkylene-NR^{4a}R^{4b}; wherein each X² is independently a bond, C(O), C₁₋₄ alkylene, C(O)—C₁₋₄ alkylene, and C₁₋₄ alkylene-C(O), and each R^{4a} and R^{4b} is independently selected from the group consisting of hydrogen, C₁₋₄ alkyl, and C₁₋₄ haloalkyl;
each R⁵ is independently selected from the group consisting of C₁₋₈ alkyl, C₁₋₈ alkoxy, C₁₋₈ haloalkyl, C₁₋₈ haloalkoxy, C₁₋₈ hydroxyalkyl, halogen, OH, CN, C(O)R^{5a} and CO₂R^{5a}; wherein each R^{5a} is independently selected from the group consisting of hydrogen, C₁₋₄ alkyl, and C₁₋₄ haloalkyl;
each R^{x} is independently selected from the group consisting of halogen, CN, C₁₋₄ alkyl, C₁₋₄ alkoxy, C₁₋₄ haloalkyl, C₁₋₄ haloalkoxy, C₁₋₄ hydroxy, C₂₋₄ alkenyl, C₃₋₆ cycloalkyl, CO₂—C₁₋₄ alkyl, and CONH₂;
the subscript m is 0, 1, 2, 3 or 4; and
the subscript n is 0, 1, 2 or 3.

In some embodiments, the compounds of formula (I) are represented by formula (IVa) or (IVb):

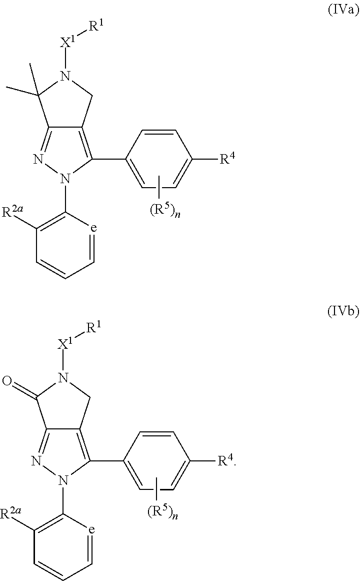

In yet another group of embodiments for the compounds of formula (IV), (IVa) or (IVb), or a pharmaceutically acceptable salt thereof, wherein R⁴ is selected from the group consisting of

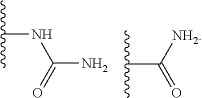

In some embodiments, the compound of Formula (IV) has the structure

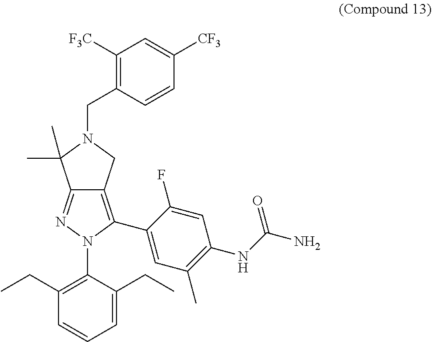 [SMILES: CCc1cccc(CC)c1-n1nc2c(c1-c1cc(C)c(NC(N)=O)cc1F)CN(Cc1ccc(C(F)(F)F)cc1C(F)(F)F)C2(C)C]

or a pharmaceutically acceptable salt thereof.

In some embodiments, the compound of Formula (IV) has the structure

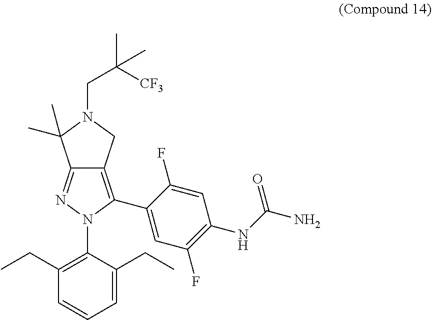 [SMILES: CCc1cccc(CC)c1-n1nc2c(c1-c1cc(F)c(NC(N)=O)cc1F)CN(CC(C)(C)C(F)(F)F)C2(C)C]

or a pharmaceutically acceptable salt thereof.

In some embodiments, the compound of Formula (IV) has the structure

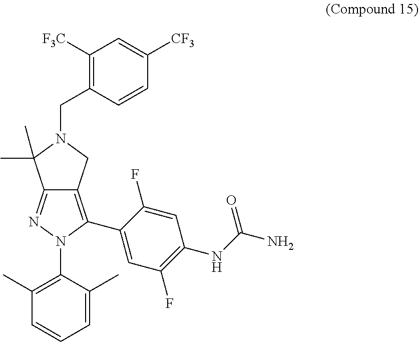 [SMILES: Cc1cccc(C)c1-n1nc2c(c1-c1cc(F)c(NC(N)=O)cc1F)CN(Cc1ccc(C(F)(F)F)cc1C(F)(F)F)C2(C)C]

or a pharmaceutically acceptable salt thereof.

In some embodiments, the compound of Formula (IV) has the structure

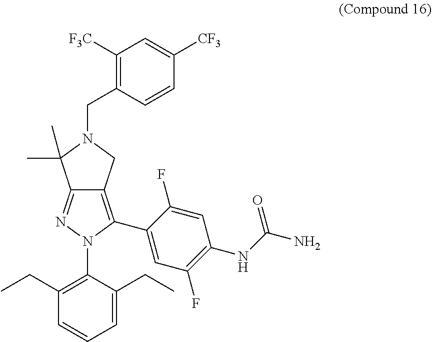 [SMILES: CCc1cccc(CC)c1-n1nc2c(c1-c1cc(F)c(NC(N)=O)cc1F)CN(Cc1ccc(C(F)(F)F)cc1C(F)(F)F)C2(C)C]

or a pharmaceutically acceptable salt thereof.

In some embodiments, the C5a inhibitor is selected from the formulas, compounds or pharmaceutical compositions disclosed in WO2019/126431 (PCT Application No. PCT/US2018/66677, filed on Dec. 20, 2018 by ChemoCentryx), the content of which is incorporated herein for all purposes.

In some embodiments, the C5a inhibitor is selected from the formulas, compounds or pharmaceutical compositions disclosed in WO2018/222601 (PCT Application No. PCT/US2018/34908, filed on May 29, 2018 by ChemoCentryx), the content of which is incorporated herein for all purposes.

In some embodiments, the C5a inhibitor is selected from the formulas, compounds or pharmaceutical compositions disclosed in WO2019/195159 (PCT Application No. PCT/US2019/25165, filed on Apr. 1, 2019 by ChemoCentryx), the content of which is incorporated herein for all purposes.

In some embodiments, the C5a inhibitor is selected from the formulas, compounds or pharmaceutical compositions disclosed in WO2017/176620 (PCT Application No. PCT/US2017/25704, filed on Apr. 3, 2017 by ChemoCentryx), the content of which is incorporated herein for all purposes.

In some embodiments, the C5a inhibitor is selected from the formulas, compounds or pharmaceutical compositions disclosed in WO2019/089468 (PCT Application No. PCT/US2018/58027, filed on Oct. 29, 2018 by ChemoCentryx), the content of which is incorporated herein for all purposes.

C. Pharmaceutical Compositions

The compounds provided herein can be administered as compositions which will typically contain a pharmaceutical carrier or diluent.

The term “composition” as used herein is intended to encompass a product comprising the specified ingredients in the specified amounts, as well as any product which results, directly or indirectly, from combination of the specified ingredients in the specified amounts.

In some embodiments, the pharmaceutical composition further comprises one or more additional therapeutic agents.

The pharmaceutical compositions for the administration of the compounds of this disclosure may conveniently be presented in unit dosage form and may be prepared by any of the methods well known in the art of pharmacy and drug delivery. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, polyethylene glycol (PEG) of various average sizes (e.g., PEG400, PEG4000) and certain surfactants such as cremophor or solutol, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono- or di-glycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present disclosure are employed. As used herein, topical application is also meant to include the use of mouth washes and gargles.

The compounds of this disclosure may also be coupled a carrier that is a suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the disclosure may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the disclosure, the compound of the disclosure is coupled to a polymer or semipermeable polymer matrix that is formed as a stent or stent-graft device.

D. Dosing & Routes of Administration

Treatment methods provided herein include, in general, administration to a patient an effective amount of one or more compounds provided herein. Suitable patients include those patients suffering from or susceptible to (i.e., prophylactic treatment) a respiratory disease or a viral infection identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

Dosage levels of the order of from about 0.1 mg to about 140 mg per kilogram of body weight per day are useful in the treatment or preventions of conditions involving pathogenic C5a activity (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. For compounds administered orally, transdermally, intravaneously, or subcutaneously, it is preferred that sufficient amount of the compound be administered to achieve a serum concentration of 5 ng (nanograms)/mL-10 μg (micrograms)/mL serum, more preferably sufficient compound to achieve a serum concentration of 20 ng-1 μg/ml serum should be administered, most preferably sufficient compound to achieve a serum concentration of 50 ng/ml-200 ng/ml serum should be administered.

Frequency of dosage may also vary depending on the compound used and the particular disease treated. However, for treatment of most disorders, a dosage regimen of 4 times daily, three times daily, or less is preferred, with a dosage regimen of once daily or 2 times daily being particularly preferred. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

### III. Examples

### Example 1: Phase 2 Study for Determining the Therapeutic Effect and Tolerance of a Small Molecule C5a Inhibitor

The objective of the trial is to determine the therapeutic effect and tolerance of Compound 2 in patients with a diagnosed respiratory illness associated with Coronavirus disease 2019 (COVID-19). Compound 2 is an inhibitor of C5aR that blocks the binding of C5a. The study has a cohort multiple Randomized Controlled Trials (cmRcT) design. Compound 2 will be administered to consenting adult patients hospitalized with COVID-19 either diagnosed with moderate or severe pneumonia requiring no mechanical ventilation or critical pneumonia requiring mechanical ventilation. Patients who will chose not to receive Compound 2 will receive standard of care. Outcomes of Compound 2 treated patients will be compared with outcomes of standard of care-treated patients as well as with outcomes of patients treated with other immune modulators.

Patients will be randomized based on their opt-in/opt-out decision to take Compound 2. This will be an open label trial, with a parallel assignment intervention model.

Patients receiving Compound 2 will take 30 mg b.i.d. for 28 days or until symptoms resolve. Compound 2 plasma levels will be monitored on days 1, 4, 8, 12, 16, 20, 24, and 28. Patients receiving standard of care will follow the suggested treatment regimen for this intervention.

The primary end points measured will be survival without need for intubation at day 14; change in organ failure at day 3 (defined by the relative variation in Sequential Organ Failure Assessment score); and change in PaO₂/FiO₂ from day 0 to day 5 of treatment as compared to patients receiving standard of care treatment.

Secondary endpoints include time to survival at days 7, 14, 28, and 90, ICU discharge, time to discharge, number of ventilator-free days through day 28 as compared to patients receiving standard of care treatment.

Inclusion criterial includes patients between 18 and 65 year old patients. Patients will be split into the starting groups: patients not requiring ICU at admission and patients requiring ICU based on severity of the COVID pneumopathy.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating a respiratory disease in a subject comprising administering to the subject an effective amount of a small molecule C5a inhibitor.

2. The method of claim 1, wherein the respiratory disease is caused by a virus.

3. The method of claim 2, wherein the virus is selected from the group consisting of an influenza virus, a coronavirus, a respiratory syncytial virus.

4. The method of claim 3, wherein the influenza virus is selected from the group consisting of influenza A virus, influenza B virus, and influenza C virus.

5. The method of claim 4, wherein the influenza A virus is selected from the group consisting of H1N1, H5N1, and H7N9.

6. The method of claim 3, wherein the coronavirus is selected from the group consisting of severe acute respiratory syndrome coronavirus (SARS-CoV), severe acute respiratory syndrome coronavirus 2 (SARS-CoV-2), and middle east respiratory syndrome (MERS) coronavirus.

7. The method of claim 1, wherein the respiratory disease is acute lung injury (ALI).

8. The method of claim 1, wherein the respiratory disease is acute respiratory distress syndrome (ARDS).

9. The method of claim 1, wherein the respiratory disease is severe acute respiratory syndrome (SARS).

10. A method of treating coronavirus disease 2019 (COVID-19) in a subject comprising administering to the subject an effective amount of a small molecule C5a inhibitor.

11. The method of any one of claims 1 to 10, wherein said small molecule C5a inhibitor is a compound of Formula (I) 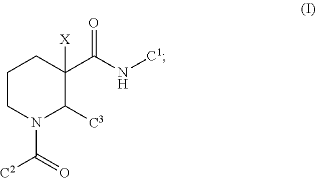
and pharmaceutically acceptable salts, hydrates and rotomers thereof, wherein
C¹ is selected from the group consisting of aryl and heteroaryl, wherein the heteroaryl group has from 1-3 heteroatoms as ring members selected from N, O and S; and wherein said aryl and heteroaryl groups are optionally substituted with from 1 to 3 R¹ substituents;
C² is selected from the group consisting of aryl and heteroaryl, wherein the heteroaryl group has from 1-3 heteroatoms as ring members selected from N, O and S; and wherein said aryl and heteroaryl groups are optionally substituted with from 1 to 3 R² substituents;
C³ is selected from the group consisting of C₁₋₈ alkyl, C₃₋₈ cycloalkyl, C₃₋₈ cycloalkyl-C₁₋₄ alkyl, aryl, aryl-C₁₋₄ alkyl, heteroaryl, heteroaryl-C₁₋₄ alkyl, heterocycloalkyl or heterocycloalkyl-C₁₋₄ alkyl, wherein the heterocycloalkyl group or portion has from 1-3 heteroatoms selected from N, O and S, and wherein the heteroaryl group has from 1-3 heteroatoms as ring members selected from N, O and S, and each C³ is optionally substituted with from 1-3 R³ substituents;
each R¹ is independently selected from the group consisting of
halogen, —CN, —R^{c}, —CO₂R^{a}, —CONR^{a}R^{b}, —C(O)R^{a}, —OC(O)NR^{a}R^{b}, —N^{b}C(O)R^{a}, —NR^{b}C(O)₂R^{c}, —NR^{a}—C(O)NR^{a}R^{b}, —NR^{a}C(O)NR^{a}R^{b}, —NR^{a}R^{b}, —OR^{a}, and —S(O)₂NR^{a}R^{b}; wherein each R^{a} and R^{b} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S; each R^{c} is independently selected from the group consisting of C₁₋₈ alkyl, C₁₋₈ haloalkyl, C₃₋₆ cycloalkyl, heterocycloalkyl, aryl and heteroaryl, and wherein the aliphatic and cyclic portions of R^{a}, R^{b} and R^{c} are optionally further substituted with from one to three halogen, hydroxy, methyl, amino, alkylamino and dialkylamino groups; and
optionally when two R¹ substituents are on adjacent atoms, are combined to form a fused five or six-membered carbocyclic ring;
each R² is independently selected from the group consisting of
halogen, —CN, —R^{f}, —CO₂R^{d}, —CONR^{d}R^{e}, —C(O)R^{d}, —OC(O)NR^{d}R^{c}, —NR^{e}C(O)R^{d}, —NR^{e}C(O)₂R^{f}, —NR^{d}C(O)NR^{d}R^{e}, —NR^{d}C(O)NR^{d}R^{e}, —N^{d}R^{e}, —OR^{d}, and —S(O)₂NR^{d}R^{e}; wherein each R^{d} and R^{c} is independently selected from hydrogen, C₁₋₈ alkyl, and C₁₋₈ haloalkyl, or when attached to the same nitrogen atom can be combined with the nitrogen atom to form a five or six-membered ring having from 0 to 2 additional heteroatoms as ring members selected from N, O or S; each R is independently selected from the group consisting of C₁₋₈ alkyl, C₁₋₈ haloalkyl, C₃₋₆ cycloalkyl, heterocycloalkyl, aryl and heteroaryl, and wherein the aliphatic and cyclic portions of R^{d}, R and R are optionally further substituted with from one to three halogen, hydroxy, methyl, amino, alkylamino and dialkylamino groups;
each R³ is independently selected from the group consisting of halogen, —CN, —Rⁱ, —CO₂R^{g}, —CONR^{g}R^{h}, —C(O)R^{g}, —OC(O)NR^{g}R^{h}, —NR^{h}C(O)R^{g}, —NR^{h}C(O)₂Rⁱ, —NR^{g}C(O)NR^{g}R^{h}, —NR^{g}R^{h}, —OR^{g}, —S(O)₂NR^{g}R^{h}, —X⁴—R^{j}, —X⁴—NR^{g}R^{h}, —X⁴—CONR^{g}R^{h}, —X⁴—NR^{h}C(O)R^{g}, —NHR^{j} and —NHCH₂R^{j}, wherein X⁴ is a C₁₋₄ alkylene; each R^{g} and R^{h} is independently selected from hydrogen, C₁₋₈ alkyl, C₃₋₆ cycloalkyl and C₁₋₈ haloalkyl, or when attached to the same nitrogen atom can be combined with the nitrogen atom to form a five or six-membered ring having from 0 to 2 additional heteroatoms as ring members selected from N, O or S and is optionally substituted with one or two oxo; each R is independently selected from the group consisting of C₁₋₈ alkyl, C₁₋₈ haloalkyl, C₃₋₆ cycloalkyl, heterocycloalkyl, aryl and heteroaryl; and each R^{j} is selected from the group consisting of C₃₋₆ cycloalkyl, pyrrolinyl, piperidinyl, morpholinyl, tetrahydrofuranyl, and tetrahydropyranyl, and wherein the aliphatic and cyclic portions of R^{g}, R^{h}, Rⁱ and R^{j} are optionally further substituted with from one to three halogen, methyl, CF₃, hydroxy, amino, alkylamino and dialkylamino groups; and
X is hydrogen or CH₃.

12. The method of claim 11, wherein said compound of Formula I has the formula selected from the group consisting of 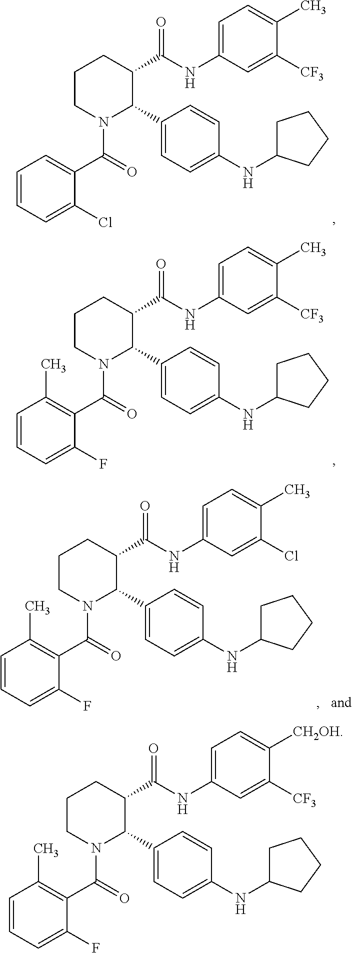 [SMILES: Cc1ccc(NC(=O)[C@H]2CCCN(C(=O)c3ccccc3Cl)[C@H]2c2ccc(NC3CCCC3)cc2)cc1C(F)(F)F ; Cc1ccc(NC(=O)[C@H]2CCCN(C(=O)c3c(C)cccc3F)[C@H]2c2ccc(NC3CCCC3)cc2)cc1C(F)(F)F ; Cc1ccc(NC(=O)[C@H]2CCCN(C(=O)c3c(C)cccc3F)[C@H]2c2ccc(NC3CCCC3)cc2)cc1Cl ; Cc1cccc(F)c1C(=O)N1CCC[C@H](C(=O)Nc2ccc(CO)c(C(F)(F)F)c2)[C@@H]1c1ccc(NC2CCCC2)cc1]
or a pharmaceutically acceptable salt thereof.

13. The method of any one of claims 1 to 10, wherein said small molecule C5a inhibitor is a compound of Formula (II) 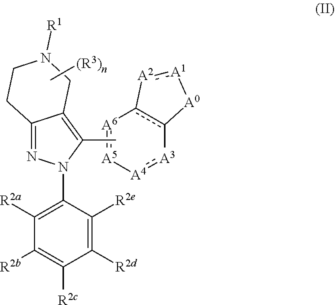
or a pharmaceutically acceptable salt thereof, wherein,
ring vertex A⁰ is NH or (O);
each of ring vertices A¹ and A³ are independently selected from the group consisting of N, NH, CH, C(O) and C(R⁴);
each of ring vertices A², A⁵ and A⁶ is independently selected from the group consisting of N, CH, and C(R⁴);
ring vertex A⁴ is selected from the group consisting of N, N(C₄ alkyl), CH, and C(R⁴);
and no more than two of A³, A⁴, A⁵ and A⁶ are N;
each of the dashed bonds independently is a single or double bond;
R¹ is selected from the group consisting of heteroaryl, C₆₋₁₀ aryl, —C₁₋₈ alkylene-heteroaryl, —C₁₋₈ alkylene-C₆₋₁₀ aryl, C₃₋₈ cycloalkyl, four to eight membered heterocycloalkyl, C₁₋₈ alkyl, C₁₋₈ haloalkyl, —C(O)NR^{1a}R^{1b}, and —CO₂R^{1a}; wherein the heterocycloalkyl group is a 4 to 8 membered ring having from 1 to 3 heteroatoms as ring vertices selected from N, O and S; the heteroaryl group is a 5 to 10 membered aromatic ring having from 1 to 3 heteroatoms as ring vertices selected from N, O and S;
wherein R^{1a} and R^{1b} are each independently selected from the group consisting of hydrogen, C₁₋₈ alkyl, C₆₋₁₀ aryl, and —C₁₋₆ alkylene-C₆₋₁₀ aryl;
wherein R¹ is optionally substituted with 1 to 5 R⁵ substituents;
R^{2a} and R^{2e} are each independently selected from the group consisting of C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, CN, and halogen;
R^{2b}, R^{2c}, and R^{2d} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, cyano, and halogen;
each R³ is independently selected from the group consisting of C₁₋₄ alkyl, C₁₋₄ haloalkyl and hydroxyl, and optionally two R³ groups on the same carbon atom are combined to form oxo (═O);
each R⁴ is independently selected from the group consisting of C₁₋₆ alkyl, C₁₋₆ alkoxy, C₁₋₆ hydroxyalkyl, C₁₋₆ haloalkyl, C₁₋₆ haloalkoxy, —O—C₁₋₆ haloalkyl, halogen, cyano, hydroxyl, —S—C₁₋₆ alkyl, —C₁₋₆ alkyl-O—C₁₋₆ alkyl, —C₁₋₆ alkyl-S—C₁₋₆ alkyl, —NR^{4a}R^{4b}, —CONR^{4a}R^{4b}, —CO₂R^{4a}, —COR^{4a}, —OC(O)NR^{4a}R^{4b}, —NR^{4a}C(O)R^{4b}, —NR^{4a}C(O)₂R^{4b}, and —NR^{4a}—C(O)NR^{4a}R^{4b};
each R^{4a} and R^{4b} is independently selected from the group consisting of hydrogen, C₁₋₄ alkyl, and C₁₋₄ haloalkyl;
each R⁵ is independently selected from the group consisting of C₁₋₈ alkyl, C₁₋₈ alkoxy, C₁₋₈ haloalkyl, C₁₋₈ haloalkoxy, C₁₋₈ hydroxyalkyl, —C₁₋₈ alkyl-heterocycloalkyl, —C₁₋₈ alkyl-C₃₋₈ cycloalkyl, C₃₋₆ cycloalkyl, heterocycloalkyl, halogen, OH, C₂₋₈ alkenyl, C₂₋₈ alkynyl, CN, C(O)R^{5a}, —NR^{5b}C(O)R^{5a}, —CONR^{5a}R^{5b}, —NR^{5a}R^{5b}, —C₁₋₈ alkylene-NR^{5a}R^{5b}, —S—C₁₋₆ alkyl, —C₁₋₆ alkyl-O—C₁₋₆ alkyl, —C₁₋₆ alkyl-S—C₁₋₆ alkyl, —OC(O)NR^{5a}R^{5b}, —NR^{5a}C(O)₂R^{5b}, —NR^{5a}—C(O)NR^{5a}R^{5b} and CO₂R^{5a}; wherein wherein the heterocycloalkyl group is a 4 to 8 membered ring having from 1 to 3 heteroatoms as ring vertices selected from N, O and S;
wherein each R^{5a} and R^{5b} is independently selected from the group consisting of hydrogen, C¹⁻⁴ alkyl, and C₁₋₄ haloalkyl, or when attached to the same nitrogen atom R^{5a} and R^{5b} are combined with the nitrogen atom to form a five or six-membered ring having from 0 to 1 additional heteroatoms as ring vertices selected from N, O, or S; and
the subscript n is 0, 1, 2 or 3.

14. The method of claim 15, wherein said compound of Formula (II) has the formula selected from the group consisting of 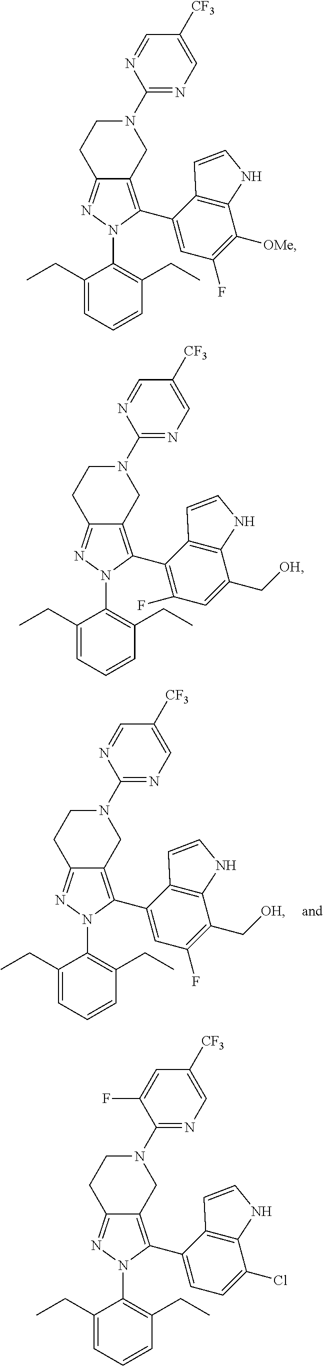 [SMILES: CCc1cccc(CC)c1-n1nc2c(c1-c1cc(F)c(OC)c3[nH]ccc13)CN(c1ncc(C(F)(F)F)cn1)CC2 ; CCc1cccc(CC)c1-n1nc2c(c1-c1c(F)cc(CO)c3[nH]ccc13)CN(c1ncc(C(F)(F)F)cn1)CC2 ; CCc1cccc(CC)c1-n1nc2c(c1-c1cc(F)c(CO)c3[nH]ccc13)CN(c1ncc(C(F)(F)F)cn1)CC2 ; CCc1cccc(CC)c1-n1nc2c(c1-c1ccc(Cl)c3[nH]ccc13)CN(c1ncc(C(F)(F)F)cc1F)CC2]
or a pharmaceutically acceptable salt thereof.

15. The method of any one of claims 1 to 10, wherein said small molecule C5a inhibitor is a compound of Formula (III) 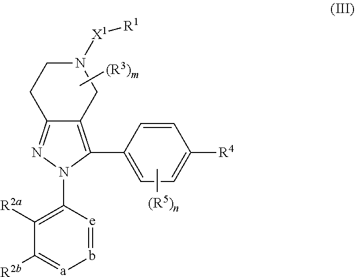
or a pharmaceutically acceptable salt thereof, wherein,
ring vertex a is N or C(R^{2c}), ring vertex b is N or C(R^{2d}), and ring vertex e is N or C(R^{2c}), wherein no more than one of a, b and e is N;
X¹ is selected from the group consisting of a bond, C₁₋₈ alkylene, C(O), C(O)—C₁₋₄ alkylene, and S(O)₂;
R¹ is selected from the group consisting of
a) 5- to 10-membered heteroaryl having from 1 to 4 heteroatoms as ring vertices selected from N, O and S;
b) C₆₋₁₀ aryl;
c) C₃₋₈ cycloalkyl;
d) 4- to 8-membered heterocycloalkyl having from 1 to 2 heteroatoms as ring vertices selected from N, O and S; and
e) C₁₋₈ alkyl, C₁₋₈ alkoxy, C₁₋₈ haloalkyl, —C(O)NR^{1a}R^{1b}, and —CO₂R^{1a}; wherein R^{1a} and R^{1b} are each independently selected from the group consisting of hydrogen, C₁₋₈ alkyl, C₆₋₁₀ aryl, and —C₁₋₆ alkylene-C₆₋₁₀ aryl;
wherein the group —X¹—R¹ is optionally substituted with 1 to 5 R^{x} substituents;
R^{2a} and R^{2e} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, CN, and halogen, and at least one of R^{2a} and R^{2e} is other than hydrogen;
R^{2b}, R^{2c}, and R^{2d} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, cyano, and halogen;
each R³ is independently selected from the group consisting of hydroxyl, C₁₋₄ alkyl, C₁₋₄ haloalkyl and C₁₋₄ hydroxyalkyl, and optionally two R³ groups on the same carbon atom are combined to form oxo (═O), and optionally two R³ groups and the carbon atoms they are attached to form a 3-6 membered ring with 0-2 hetereoatoms as ring members selected from O, N, and S;
R⁴ is independently selected from the group consisting of X²—OR^{4a}, —X²NR^{4a}R^{4b}, —X²—CONR^{4a}R^{4b}, —X²—NR^{4a}—C(O)R^{4a}, —X²—NR^{4a}—C(O)NR^{4a}R^{4b}, —X²—NR^{4a}—C(O)OR^{4a}, —X²—NR^{4a}—C(O)—C₁₋₃ alkylene-OR^{4a} and —X²—NR^{4a}—C(O)—C₁₋₃ alkylene-NR^{4a}R^{4b}; wherein each X² is independently a bond, C(O), C₁₋₄ alkylene, C(O)—C₁₋₄ alkylene, and C₁₋₄ alkylene-C(O), and each R^{4a} and R^{4b} is independently selected from the group consisting of hydrogen, C₁₋₄ alkyl, and C₁₋₄ haloalkyl;
each R⁵ is independently selected from the group consisting of C₁₋₈ alkyl, C₁₋₈ alkoxy, C₁₋₈ haloalkyl, C₁₋₈ haloalkoxy, C₁₋₈ hydroxyalkyl, halogen, OH, CN, C(O)R^{5a} and CO₂R^{5a}; wherein each R^{5a} is independently selected from the group consisting of hydrogen, C₁₋₄ alkyl, and C₁₋₄ haloalkyl;
each R^{x} is independently selected from the group consisting of halogen, CN, C₁₋₄ alkyl, C₁₋₄ alkoxy, C₁₋₄ haloalkyl, C₁₋₄ haloalkoxy, C₁₋₄ hydroxy, C₂₋₄ alkenyl, C₃₋₆ cycloalkyl, CO₂—C₁₋₄ alkyl, and CONH₂;
the subscript m is 0, 1, 2, 3 or 4; and
the subscript n is 0, 1, 2 or 3.

16. The method of claim 15, wherein said compound of Formula (III) has the formula selected from the group consisting of 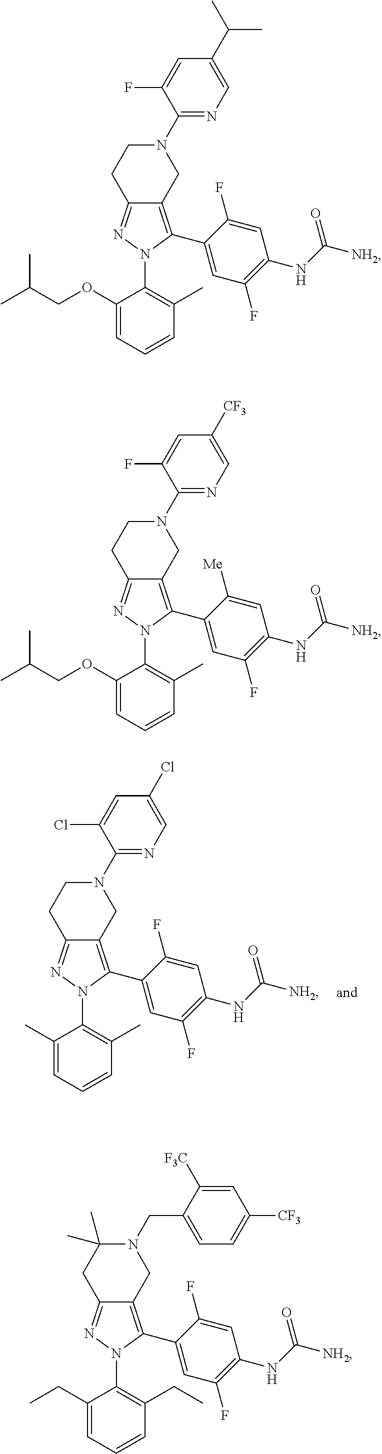
or a pharmaceutically acceptable salt thereof.

17. The method of any one of claims 1 to 10, wherein said small molecule C5a inhibitor is a compound of Formula (IV) 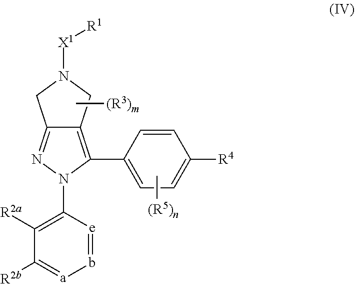
or a pharmaceutically acceptable salt thereof, wherein,
ring vertex a is N or C(R^{2c}), ring vertex b is N or C(R^{2d}), and ring vertex e is N or C(R^{2c}), wherein no more than one of a, b and e is N;
X¹ is selected from the group consisting of a bond, C₁₋₈ alkylene, C(O), C(O)—C₁₋₄ alkylene, and S(O)₂;
R¹ is selected from the group consisting of
a) 5- to 10-membered heteroaryl having from 1 to 4 heteroatoms as ring vertices selected from N, O and S;
b) C₆₋₁₀ aryl;
c) C₃₋₈ cycloalkyl;
d) 4- to 8-membered heterocycloalkyl having from 1 to 2 heteroatoms as ring vertices selected from N, O and S; and
e) C₁₋₈ alkyl, C₁₋₈ alkoxy, C₁₋₈ haloalkyl, —C(O)NR^{1a}R^{1b}, and —CO₂R^{1a}; wherein R^{1a} and R^{1b} are each independently selected from the group consisting of hydrogen, C₁₋₈ alkyl, C₆₋₁₀ aryl, and —C₁₋₆ alkylene-C₆₋₁₀ aryl;
wherein the group —X¹—R¹ is optionally substituted with 1 to 5 R^{x} substituents;
R^{2a} and R^{2e} are each independently selected from the group consisting of hydrogen, C₁₋₆ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, CN, and halogen, and at least one of R^{2a} and R^{2e} is other than hydrogen;
R^{2b}, R^{2c}, and R^{2d} are each independently selected from the group consisting of hydrogen, C₁₋₄ alkyl, C₁₋₆ alkoxy, C₁₋₆ haloalkyl, —O—C₁₋₆ haloalkyl, —S—C₁₋₆ alkyl, —C₁₋₆ alkyl-O—C₁₋₆ alkyl, —C₁₋₆ alkyl-S—C₁₋₆ alkyl, cyano, and halogen;
each R³ is independently selected from the group consisting of hydroxyl, C₁₋₄ alkyl, C₁₋₄ haloalkyl and C₁₋₄ hydroxyalkyl, and optionally two R³ groups on the same carbon atom are combined to form oxo (═O), and optionally two R³ groups and the carbon atoms they are attached to form a 3-6 membered ring with 0-2 hetereoatoms as ring members selected from O, N, and S;
R⁴ is independently selected from the group consisting of —X²—OR^{4a}, —X²NR^{4a}R^{4b}, —X²—CONR^{4a}R^{4b}, —X²—NR^{4a}—C(O)R^{4a}, —X²—NR^{4a}—C(O)NR^{4a}R^{4b}, —X²—NR^{4a}—C(O)OR^{4a}, —X²—NR^{4a}—C(O)—C₁₋₃ alkylene-OR^{4a} and —X²—NR^{4a}—C(O)—C₁₋₃ alkylene-NR^{4a}R^{4b}; wherein each X² is independently a bond, C(O), C₁₋₄ alkylene, C(O)—C₁₋₄ alkylene, and C₁₋₄ alkylene-C(O), and each R^{4a} and R^{4b} is independently selected from the group consisting of hydrogen, C₁₋₄ alkyl, and C₁₋₄ haloalkyl;
each R⁵ is independently selected from the group consisting of C₁₋₈ alkyl, C₁₋₈ alkoxy, C₁₋₈ haloalkyl, C₁₋₈ haloalkoxy, C₁₋₈ hydroxyalkyl, halogen, OH, CN, C(O)R^{5a} and CO₂R^{5a}; wherein each R^{5a} is independently selected from the group consisting of hydrogen, C₁₋₄ alkyl, and C₁₋₄ haloalkyl;
each R^{x} is independently selected from the group consisting of halogen, CN, C₁₋₄ alkyl, C₁₋₄ alkoxy, C₁₋₄ haloalkyl, C₁₋₄ haloalkoxy, C₁₋₄ hydroxy, C₂₋₄ alkenyl, C₃₋₆ cycloalkyl, CO₂—C₁₋₄ alkyl, and CONH₂;
the subscript m is 0, 1, 2, 3 or 4; and
the subscript n is 0, 1, 2 or 3.

18. The method of claim 17, wherein said compound of Formula (IV) has the formula selected from the group consisting of 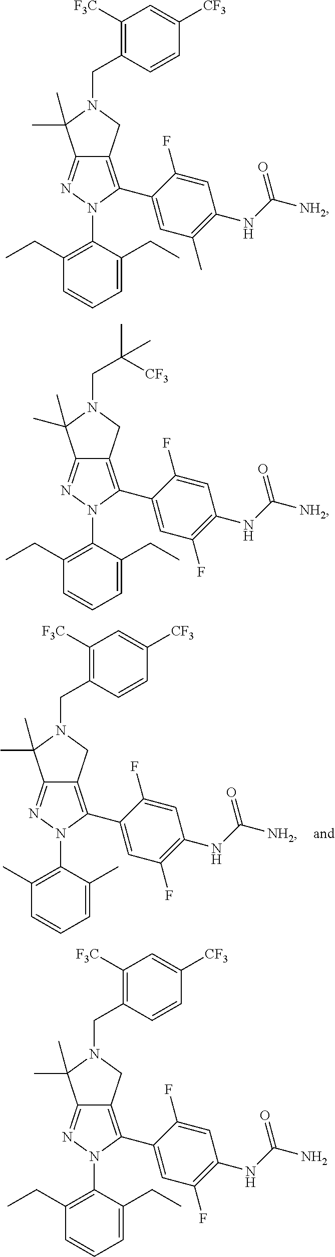
or a pharmaceutically acceptable salt thereof.
